(19) United States
(12) Patent Application Publication
(10) Pub. No.: US 2005/0143400 A1
(43) Pub. Date: Jun. 30, 2005

(54) Process for preparing famciclovir

(76) Inventors: Genny Shamai, Kiryat Yam (IL); Shlomo Antebi, Haifa (IL); David Ioffe, Haifa (IL); Ben-Zion Dolitzky, Petach Tiqva (IL); Batia Kauffmann, Carmiel (IL)

Correspondence Address: KENYON & KENYON, ONE BROADWAY, NEW YORK, NY, 10004, US

(21) Appl. No.: 10/932,120

(22) Filed: Sep. 2, 2004

Related U.S. Application Data
(60) Provisional application No. 60/500,575, filed on Sep. 4, 2003.

(51) Int. Cl. A61K 31/522; C07D 473/14

(52) U.S. Cl. 514/263.38; 544/276

(57) ABSTRACT

The invention provides a process for making famciclovir, comprising reacting 9-[4-acetoxy-3-(acetoxymethyl)but-1-yl]-2-amino-6-chloropurine (Cl-FMC) with a palladium on charcoal catalyst in water and ammonium formate. The invention also provides methods of treating viral diseases by administering the famciclovir prepared according to the above process.

### CROSS REFERENCE TO RELATED APPLICATION

The present application claims the benefit of U.S. Provisional Application No. 60/500,575 filed on Sep. 4, 2003, the disclosure of which is incorporated by reference.

### FIELD OF THE INVENTION

The present invention relates to a process for making famciclovir containing low levels of by-products, famciclovir containing low levels of by-products prepared by such a process, pharmaceutical formulations comprising famciclovir containing low levels of by-products, and a method of treating a viral disease comprising administering famciclovir containing low levels of by-products.

### BACKGROUND OF THE INVENTION

Famciclovir, available as Famvir®, is an antiviral drug developed by SmithKline Beecham. Famciclovir, administered orally, is indicated for the treatment of acute herpes zoster (shingles). It is also indicated for the treatment or suppression of recurrent genital herpes in immunocompetent patients and for the treatment of recurrent mucocutaneous herpes simplex infections in HIV infected patients. Famciclovir has the following chemical formula:

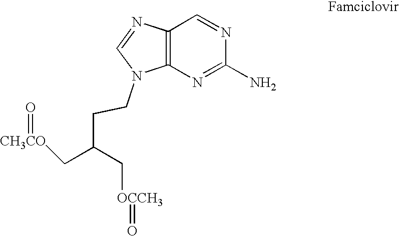

The chemical name for famciclovir is 2-[2-(2-amino-9H-purin-9-yl)ethyl]-1,3-propanediol diacetate. Famciclovir is reported to be a white to pale yellow solid freely soluble in acetone and methanol, but sparingly soluble in ethanol and isopropanol. At 25° C., the anhydrous form of famciclovir dissolves freely in water (>25% w/v) and rapidly precipitates as a sparingly soluble monohydrate (2-3% w/v). Below 85% relative humidity, famciclovir is not hygroscopic. Its partition coefficients are reported to be: octanol/water (pH 4.8) P=1.09 and octanol/phosphate buffer (pH 7.4) P=2.08.

U.S. Pat. No. 5,246,937 discloses that famciclovir may be produced by the hydrogenolysis of a compound of formula I (acetic acid 2-acetoxymethyl-4-(5-amino-7-chloro-imidazo[4,5-b]pyridin-3-yl)-butyl ester (Cl-FMC)) in a palladium on charcoal (Pd/C) catalyst in methanol containing ammonium formate:

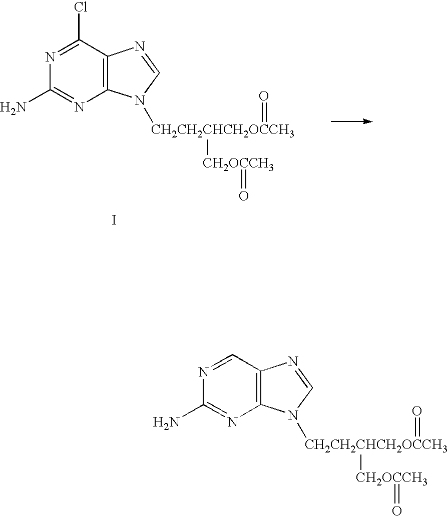

However, this process leads to high levels of two impurities:

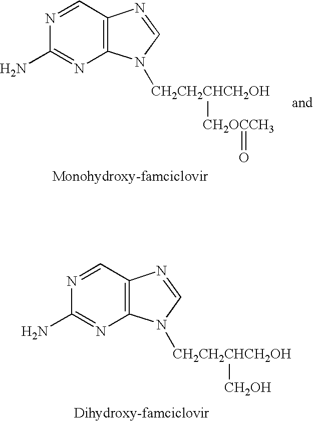

Thus, there remains a need for a process for producing famciclovir in high yields and with low levels of undesirable by-products.

### SUMMARY OF THE INVENTION

The present invention provides a process for producing famciclovir with low levels of undesirable by-products, which process comprises reacting a compound of formula I, acetic acid 2-acetoxymethyl-4-(5-amino-7-chloro-imidazo[4,5-b]pyridin-3-yl)-butyl ester, in the presence of a palladium catalyst and water.

Specifically, the present invention provides a process of preparing famciclovir comprising the steps of:
a) providing a suspension comprising acetic acid 2-acetoxymethyl-4-(5-amino-7-chloro-imidazo[4,5-b]pyridin-3-yl)-butyl ester (Cl-FMC), a palladium catalyst, and water, e.g. by mixing acetic acid 2-acetoxymethyl-4-(5-amino-7-chloro-imidazo[4,5-b]pyridin-3-yl)-butyl ester, the palladium catalyst and water to form the suspension;
b) adding a solution of ammonium formate to the suspension; and
c) isolating famciclovir.

Preferably, the solution of ammonium formate is added dropwise to the suspension in step b).

In between steps a) and b), the above process optionally includes a step of forming the solution of ammonium formate.

Preferably, the palladium catalyst is palladium on charcoal. Preferably, the palladium on charcoal catalyst has about 3% to about 15%, more preferably about 5% to about 10%, by weight of palladium in terms of the combined weight of Pd and charcoal. Much more preferably, the palladium on charcoal catalyst has about 10% to about 25% by weight of palladium in terms of the combined weight of Pd and charcoal.

Preferably, the palladium on charcoal catalyst is wet (i.e., containing water as a solvent). More preferably, the palladium on charcoal catalyst contains about 30% to 70%, further more preferably about 40% to about 60%, and much more preferably about 50%, by weight of water, in terms of the combined weight of palladium, charcoal and water.

Preferably, the acetic acid 2-acetoxymethyl-4-(5-amino-7-chloro-imidazo[4,5-b]pyridin-3-yl)-butyl ester (Cl-FMC) and the ammonium formate are used in equimolar amount in the process of the invention, so that at the end of step b) of the above process equimolar amounts of 2-acetoxymethyl-4-(5-amino7-chloro-imidazo[4,5-b]pyridin-3-yl)-butyl ester and ammonium formate have reacted.

Preferably, the suspension comprising acetic acid 2-acetoxymethyl-4-(5-amino-7-chloro-imidazo[4,5-b]pyridin-3-yl)-butyl ester (Cl-FMC), a palladium catalyst, and water in step a) of the process of the invention is preheated to a temperature of about 40° C. or less, more preferably ranging from about 30° C. to about 40° C., much more preferably ranging from about 30° C. to about 35° C., before the addition of ammonium formate in step b). For instance, the suspension in step a) is preheated to a temperature of about 30° C. or about 35° C. Alternatively, the suspension in step a) is maintained at room temperature, e.g. about 20° C. to about 25° C., and the palladium on charcoal catalyst has less than about 10% by weight of palladium in terms of the combined weight of palladium and charcoal.

Preferably, the ammonium formate is added dropwise to the suspension in step b). More preferably, the dropwise addition is performed for at least about 2.5 hours. Even more preferably, the dropwise addition is performed for about 6 hours.

Another object of the present invention is directed toward a famciclovir prepared according to the process described above.

The famciclovir produced by the process of the present invention contains low levels of the monohydroxy-famciclovir (MH-FMC) impurity, preferably less than 1.0% (wt/wt) MH-FMC.

The present invention also provides a pharmaceutical composition comprising the famciclovir prepared according to the process of the invention and a pharmaceutically acceptable carrier or excipient.

Famciclovir prepared according to the process described above may be used in treating a viral disease in a subject, comprising administering an anti-viral effective amount of the famciclovir to the subject. Preferably, the subject is a human. The viral disease includes herpes zoster, genital herpes and mucocutaneous herpes simplex.

### DETAILED DESCRIPTION OF THE INVENTION

As used herein, the term “equimolar amounts” refers to a molar ratio of about 0.95 to about 1.05 between two reactants.

The process of the present invention for preparing famciclovir comprises reacting a compound of formula I, acetic acid 2-acetoxymethyl-4-(5-amino-7-chloro-imidazo[4,5-b]pyridin-3-yl)-butyl ester (Cl-FMC), in the presence of a palladium catalyst and water, followed by addition of ammonium formate. Preferably, the palladium catalyst is palladium on charcoal. More preferably, the palladium on charcoal is wet, i.e., the palladium on charcoal contains water. Even more preferably, the wet palladium on charcoal is heated to a temperature of about 40° C. or less, e.g. about 35° C. to about 40° C., before reacting with the solution of ammonium formate.

Preferably, a mixture of wet 10% palladium (based on the weight of Pd+charcoal) on charcoal catalyst and Cl-FMC in water is first prepared and preheated at a temperature of about 40° C. or less before the reaction with ammonium formate in the process of the present invention. Ammonium formate is highly soluble in water, therefore, strong foaming is observed during the reaction caused by evolution of carbon dioxide. The ammonium formate is preferably first prepared as a solution in water. The prepared ammonium formate solution can be added dropwise to the preheated slurry of the wet 10% palladium on charcoal catalyst and Cl-FMC in water. Under these conditions, the MH-FMC level will be reduced.

When the reaction temperature of the mixture and ammonium formate is higher than 60° C. (e.g. 64° C.), there is a high level of MH-FMC formed. Thus, preferably, the reaction temperature should be below 60° C. More preferably, the reaction temperature is 40° C. or less. Most preferably, the reaction temperature is 30-35° C., e.g. 30° C., 32.5° C. and 35° C. Alternatively, the reaction may be performed at room temperature, e.g. about 20° C. to about 25° C., but with a prolonged time (e.g. 6 hours or more) to allow the dissolution of Cl-FMC.

The impurity level of MH-FMC may further be reduced by regulating the amounts of ammonium formate and Pd/C loading. Equimolar amounts of ammonium formate and Cl-FMC lead to a low level of MH-FMC. Preferably, the Pd/C loading should be less than 10% Pd (based on the weight of Pd+C).

The above-mentioned process of the invention can provide FMC at a high yield (e.g., higher than 80%) with a low level of monohydroxy famciclovir, e.g. less than about 1.0% (wt/total wt) MH-FMC. Preferably, a second crystallization or trituration in water can give rise to famciclovir with any impurity less than 0.05 weight %.

There are several advantages of using palladium on charcoal catalyst in the presence of water. First, use of water as a solvent is environmentally safe and the process can avoid using organic solvents. Second, the reaction leads to production of FMC polymorph I directly. Third, the use of water as a solvent yields a FMC product that is significantly whiter. Fourth, the process described above yields famciclovir having low levels of the monohydroxy and dihydroxy FMC impurities.

Having thus described the various aspects of the present invention, the following examples are provided to illustrate specific embodiments of the present invention. They are not intended to be limiting in any way.

### EXAMPLES

Note: The composition of reaction mixture and solids are given as area % HPLC.

### Example 1

### Preparation of Acetic Acid 2-acetoxymethyl-4-(2-amino-purin-9-yl)-butyl Ester (FMC) from Acetic Acid 2-acetoxymethyl-4-(5-amino-7-chloro-imidazo[4,5-b]pyridin-3-yl)-butyl Ester (Cl-FMC)

A mixture of 6.2 g wet “10% Pd/C” (wt Pd/wt Pd+C) with 52.14% H₂O (wt H₂O/wt of Pd+C+H₂O), H₂O (120 ml) and Cl-FMC (30 g; 83.1 mmol) was added, under an inert atmosphere of nitrogen, into a jacketed reactor equipment with a mechanical stirrer, a reflux condenser and a thermocouple. The mixture was heated to 42° C. A solution of ammonium formate (6.5 g; 99.7 mmol; 20% excess) in 20 ml H₂O was added dropwise for 2.5 hours. After 30 min., charcoal (3 g) was added and the solution was continued to be stirred for an additional time of 30 min. The reaction mixture was filtered, and the catalyst was washed with 10 ml H₂O. The filtrate was stirred for 2 hours in an ice bath (2° C.). The precipitated solid was filtered and washed with 15 ml cold H₂O, leaving 31.5 g wet solid precipitate. Upon drying, 22.4 g of a very white solid was obtained (83.6% of the expected). The MH-FMC level was 0.29% and the FMC yield was 83.5% (HPLC area %).

### Example 2

### Preparation of Acetic Acid 2-acetoxymethyl-4-(2-amino-purin-9-yl)-butyl Ester (FMC) from Acetic Acid 2-acetoxymethyl-4-(5-amino-7-chloro-imidazo[4,5-b]pyridin-3-yl)-butyl Ester (Cl-FMC)

A mixture of 6.2 g wet “10% Pd/C” (based on the weight of Pd+C) with 52.14% H₂O (wt H₂O/wt of Pd+C+H₂O), H₂O (120 ml) and Cl-FMC (30 g; 83.1 mmol) was added, under an inert atmosphere of nitrogen, into a jacketed reactor equipment with a mechanical stirrer, a reflux condenser and a thermocouple. The mixture was preheated to 35° C. A solution of ammonium formate (5.4 g; 83.1 mmole; 8.4% in excess) in 20 ml H₂O was added dropwise for 2.5 hours. After 30 min., charcoal (3 g) was added and the solution was stirred for 30 min. The reaction mixture was filtered, and the catalyst obtained was washed with 10 ml H₂O. The filtrate was stirred for 2 hours in an ice bath (2° C.). The precipitated solid was filtered and washed with 15 ml cold H₂O, leaving 31.5 g wet solid precipitate. Upon drying, 22.4 g of a very white solid was obtained (81.3% of the expected). The MH-FMC level was 0.27% and the Cl-FMC was 0.08% (HPLC area %). All other impurities levels were less than 0.06% (HPLC area %).

### Example 3

### Preparation of Acetic Acid 2-acetoxymethyl-4-(2-amino-purin-9-yl)-butyl Ester (FMC)_from Acetic Acid 2-acetoxymethyl-4-(5-amino-7-chloro-imidazo[4,5-b]pyridin-3-yl)-butyl Ester (Cl-FMC)

Into a jacketed reactor equipment with a mechanical stirrer, a reflux condenser and a thermocouple, under an inert atmosphere (N₂), a mixture of wet “10% Pd/C” (6.2 g, wherein the 10% is based on the combined weight of Pd and C, having 52.14% H₂O (wt of H₂O/wt of P+C+H₂O)), H₂O (120 ml) and Cl-FMC (30 g; 83.1 mmol) was added. The mixture was maintained at room temperature. A solution of ammonium formate (5.4 g; 83.1 mmole; 8.4% in excess) in 20 ml H₂O was added dropwise for 6 hours. After 30 min., charcoal (3 g) was added and the solution was stirred for 30 min. The reaction mixture was filtered, and the catalyst was washed with 10 ml H₂O. The filtrate was stirred for 2 hours in an ice bath (2° C.). The precipitated solid was filtered and washed with 15 ml cold H₂O, leaving 31.5 g wet solid precipitate. Upon drying, 22.4 g of a very white solid was obtained (81.3% of the expected). The MH-FMC level was 0.27% and the Cl-FMC was 0.08% (HPLC area %). All other impurities were less than 0.06% (HPLC area %).

The purity of famciclovir was analyzed by HPLC under the following conditions:
Pumping system: HP model 1050
Detector: HP model 1100, 1=309 nm
Flow: 1.2 ml/min
Injection Volume: 20 ml
Column: ACE, C-18, 250*4.6 mm*5 mm
Solvents: A: H₂O(AmAc 0.1M)

B: CH₃CN

| Time | Solvent A | Solvent B |
|---|---|---|
| 0 | 85 | 15 |
| 5 | 85 | 15 |
| 10 | 50 | 50 |
| 20 | 50 | 50 |
| 25 | 85 | 15 |

1. A process of preparing famciclovir, comprising the steps of:
a) providing a suspension comprising 9-[4-acetoxy-3-(acetoxymethyl)but-1-yl]-2-amino-6-chloropurine (Cl-FMC), a palladium catalyst, and water;
b) adding a solution of ammonium formate to the suspension; and
c) isolating famciclovir.

2. The process of claim 1, wherein the solution of ammonium formate is an aqueous solution.

3. The process of claim 1, wherein the palladium catalyst is palladium on charcoal.

4. The process of claim 3, wherein the palladium on charcoal has about 3% to about 15% by weight of palladium in terms of the combined weight of Pd and charcoal.

5. The process of claim 4, wherein the palladium on charcoal has about 5% to about 10% by weight of palladium in terms of the combined weight of Pd and charcoal.

6. The process of claim 5, wherein the palladium on charcoal has about 10% by weight of palladium in terms of the combined weight of Pd and charcoal.

7. The process of claim 3, wherein the palladium on charcoal contains water.

8. The process of claim 7, wherein the palladium on charcoal contains about 30% to about 70% by weight of water, in terms of the combined weight of palladium, charcoal and water.

9. The process of claim 8, wherein the palladium on charcoal contains about 40% to about 60% by weight of water, in terms of the combined weight of palladium, charcoal and water.

10. The process of claim 9, wherein the palladium on charcoal contains about 50% by weight of water, in terms of the combined weight of palladium, charcoal and water.

11. The process of claim 5, wherein the palladium on charcoal contains water.

12. The process of claim 11, wherein the palladium on charcoal contains about 30% to about 70% by weight of water, in terms of the combined weight of palladium, charcoal and water.

13. The process of claim 12, wherein the palladium on charcoal contains about 40% to about 60% by weight of water, in terms of the combined weight of palladium, charcoal and water.

14. The process of claim 13, wherein the palladium on charcoal contains about 50% by weight of water, in terms of the combined weight of palladium, charcoal and water.

15. The process of claim 1, wherein the ammonium formate is added dropwise to the suspension in step b).

16. The process of claim 15, wherein the dropwise addition of the solution of ammonium formate in step b) is performed for at least about 2.5 hours.

17. The process of claim 16, wherein the dropwise addition of the solution of ammonium formate in step b) is performed for about 6 hours.

18. The process of claim 1, further comprising cooling the suspension after step b).

19. The process of claim 1, wherein equimolar amounts of the 9-[4-acetoxy-3-(acetoxymethyl)but-1-yl]-2-amino-6-chloropurine (Cl-FMC) and the ammonium formate are reacted in step b).

20. The process of claim 1, wherein the suspension in step a) is heated to a temperature of no more than about 40° C. before the addition of ammonium formate in step b).

21. The process of claim 20, wherein the suspension in step a) is heated to a temperature of ranging from about 30° C. to about 40° C. before the addition of ammonium formate in step b).

22. The process of claim 21, wherein the suspension in step a) is heated to a temperature of ranging from about 30° C. to about 35° C. before the addition of ammonium formate in step b).

23. The process of claim 3, wherein the suspension in step a) is maintained at room temperature and the palladium on charcoal has less than about 10% by weight of palladium in terms of the combined weight of palladium and charcoal.

24. The process of claim 1, wherein the suspension in step a) is heated to a temperature of less than about 60° C. before the addition of ammonium formate in step b).

25. Famciclovir prepared by the process of any one of claims 1-24.

26. A method for treating a viral disease in a subject, comprising administering an anti-viral effective amount of the famiciclovir of claim 25 to the subject, wherein the viral disease is herpes zoster, genital herpes or mucocutaneous herpes simplex.
